(19) United States
(12) Patent Application Publication
(10) Pub. No.: US 2005/0043693 A1
(43) Pub. Date: Feb. 24, 2005

(54) Easy drop

(76) Inventor: Angelo Michael Infantolino, Pine Brook, NJ (US)

Correspondence Address: AngelpoInfantolino, 351 Potomac Drive, Basking Ridge, NJ, 07920, US

(21) Appl. No.: 10/403,452

(22) Filed: Mar. 31, 2003

(51) Int. Cl. A61M 35/00

(52) U.S. Cl. 604/300

(57) ABSTRACT

Easy drop comprises a front and back side, a bottom side, atop side, and two side ends, a dropper that is attached in the top side within the aperture, a cap that is twisted on over the dropper to assure the ophthalmic solution remains in the plastic dropper bottle when not in use; and having two indentations molded within, each indentation molded within each top portion of the two side ends and within each top portions of the front and back sides, the two indentations preferably have a diameter between approximately 8 mm, and preferably are separated between approximately 2 and 12 mm; such that easy drop will enable a user to rest the plastic dropper bottle on the bridge of the nose within either indentation, and to position the dropper above the eye without touching the eye, further enabling a user to dispense a ophthalmic solution in either eye with a steady hand, independently.

### FIELD OF THE INVENTION.

The present invention relates to the field of ophthalmology, and more particularly to easy drop, a plastic dropper bottle having two indentations molded within, enabling an individual to rest easy drop on the bridge of the nose, further enabling an individual to dispense ophthalmic solutions in either eye with a steady hand, independently.

### BACKGROUND OF THE INVENTION

There is a growing trend in the ophthalmology community to seek safer, more efficient and convenient methods for dispensing ophthalmic solutions, in recent years there has been many attempts to resolve this issue.

The prior art discloses a variety of devices for the application and dispensing of ophthalmic solutions.

U.S. Pat. No. 5,201,726 (Kirkham) shows an eye bath device which delivers a spray in a generally horizontal stream and includes a cup which surrounds the eye to confine the stream to the vicinity of the eye.

U.S. Pat. No. 4,605,398 (Herrick) describes an eye dropper having an averting member to keep the lower eye lid from blocking the eye while the drops are directed into the eye.

U.S. Pat. No. 5,366,448 (Basilice) shows a eye drop delivery system which includes guides and dispensers which tend to direct the solution in the form of a spray rather than eye drops, such a spray generally includes a horizontal component of travel.

U.S. Pat. No. 5,154,710 (Williams) and U.S. Pat. No. 5,154,711 (Williams) show ophthalmic delivery devices that include an eye cup having a aperture through the cup which allows the user to look at a target external to the eye cup while the eye drop is dispensed.

Design Pat. Des. No. 330,418 (Carter); U.S. Pat. No. 4,543,096 (Keene); U.S. Pat. No. 4,733,802 (Sheldon); U.S. Pat. No. 4,960,407 (Cope); U.S. Pat. No. 5,037,406 (Smith); U.S. Pat. No. 5,178,613 (Gibilisco); and U.S. Pat. No. 5,382,243 (Mulholland) describe eye droppers which utilize an eye cup as well as a dispenser that is generally in the form of a squeezable bottle.

U.S. Pat. No. 4,338,936 (Nelson) shows a device and method for delivering finely divided solid medication to the eye utilizing a squeeze bottle. The solid material is directed horizontally at the eye and quickly dissolves in the tear fluid to produce the proper dosage necessary for an eye treatment.

U.S. Pat. No. 5,387,202 (Baron) shows an eye drop dispensing device that consists essentially of a flexible tube of oval cross section that is placed over the body of a pliable ophthalmic solution container. Located at the base of the oval tube is a rim to aid in holding a eyelid in place during the application of the medication. The tube and the enclosed pliable ophthalmic solution bottle, is simultaneously squeezed to apply the solution to the eye while the user is looking along the oval channel of the device. This device must be repeatedly removed and reattached in order to access the bottle cap.

U.S. Pat. No. (Lelicoff) teaches a device that includes a ring for attaching the device to a bottle, an end piece for engaging a user's eyelid during the application of ophthalmic solution and an inwardly curved extension piece attached to a boss on the ring. The curved extension piece is designed to aid the placement of applied drops of medicated solution. The eye engaging portion of this device interferes with easy removal of the bottle cap.

U.S. Pat. No. 6,098,676 (Phillips) teaches a drug delivery device that has a reservoir holding medicine for delivery to a patient. The device has a conduit with one end coupled to the reservoir and a free end to position within the fornix of a patients eye. Through gravity and capillary action the medicine flows into the eye with the rate of delivery adjusted according to the size and material of the conduit. The reservoir is made of an absorbent material provided with an impermeable backing which acts as a barrier, and in the preferred embodiment the backing has an adhesive for attaching to the eye of the patient.

U.S. Pat. No. 5,799,837 (Firestone, et al.) is for a packaged pharmaceutical product having an extended shelf life and includes a container consisting of a hollow body with an open end. The body wall thickness enables drop-by-drop dispensing of the medicine by manually squeezing the container body. A tip is fixed to the body to form droplets for application.

U.S. Pat. No. 5,578,020 (Mosley) is for an eye dropper dispenser and dispensing sleeve. The dropper has a liquid reservoir portion and a dispensing end with a dropper orifice. Part of the reservoir is resilient, and a dispensing sleeve circumscribes the dropper tube with a pair of legs that extend beyond the end of the tube. The legs are adopted to fit against the orbital areas of an eye to support the dropper over the eye for application of the liquid.

U.S. Pat. No. 6,371,945 (Sherman) illustrates a device that aids in the placement of eye drops from an ophthalmic medication containing bottle. The device is designed to be attached to the bottle at all times, and is specifically designed to aid in the easy removal and replacement of ophthalmic solution containing bottle caps.

U.S. Pat. No. 4,257,417 (Gibilisco) discloses a bottle holder having a nose rest and a bar with an opening through which the tip of the dispenser is mounted to a slidably position and holds the dispenser over the eye

There has been many attempts to resolve the issue of dispensing ophthalmic solutions, while the prior art discloses a variety of ophthalmic application devices, there still is not a way for an individual to dispense ophthalmic solutions easily, safely, efficiently and conveniently with a steady hand without using an extra device or placing a device on the eye or to close to the eye in which such a device could be infectious and/or dangerous to an individual's eye, respectfully to the prior art. The present invention solves this problem by providing easy drop, a plastic dropper bottle, having two indentations molded within, enabling an individual to rest easy drop on the bridge of the nose, and to position the dropper above the eye without touching the eye, further enabling an individual to dispense ophthalmic solutions in either eye with a steady hand independently, as well as being easy, safe, efficient, and more convenient.

### BRIEF SUMMARY OF THE INVENTION

It is an object of the present invention to provide easy drop, a plastic dropper bottle that will enable its users to rest easy drop on the bridge of the nose.

It is another object of the present invention to provide easy drop, a plastic dropper bottle that will enable its users to dispense ophthalmic solutions in either eye with a steady hand, independently.

Still another object of the present invention is to provide easy drop, a plastic dropper bottle that will enable its users to dispense ophthalmic solutions in either eye without touching the eye.

Yet another object of the present invention is to provide easy drop, a plastic dropper bottle that is economical to manufacture.

The present invention easy drop, a plastic dropper bottle comprising a front and back side, a bottom side, a top side and two side ends, a dropper that is attached in the top side within the aperture, including a cap that is twisted within the threads on the outside of the top side over the dropper to assure the ophthalmic solution remains in the plastic dropper when not in use; and having two indentations molded within, each indentation is molded within each top portion of each side end and within the top portions of the front and back sides. The two indentations preferably have a diameter between approximately 8 mm, and the indentations preferably being separated between approximately 2 and 12 mm, enabling a user to rest easy drop on the bridge of the nose, further enabling a user to dispense the ophthalmic solution in either eye with a steady hand, independently.

### BRIEF DESCRIPTION OF THE SEVERAL VIEWS OF THE DRAWING

FIG. 1 is a exploded cross sectional view of easy drop, the dropper and cap are separated from the plastic bottle of the present invention.

FIG. 2 is a plan view of the back of easy drop in a application position slightly angled of the present invention.

### DETAILED DESCRIPTION OF THE INVENTION

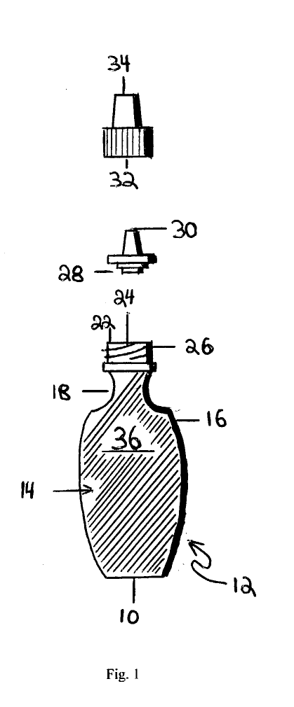

Referring now to the figures, the present invention is a plastic dropper bottle 12 in which a indentation 18 is molded within the top portion of each side end 16 and within the top portions of the front side 14 and back side 36. Plastic dropper bottle 12 comprises a front side 14 and a back side 36, a bottom side 10, a top side 22, and two side ends 16, a dropper 30, that is attached in the top side 22 within the aperture 24, including a cap 34 that is twisted within the threads 26 on the outside of the top side 22 over the dropper 30 to assure the ophthalmic solution remains in the plastic dropper bottle 12 when not in use. Reference numeral 28 refers to the bottom portion of the dropper 30 that holds the dropper 30 in the correct position when inserted in the top side 22 within the aperture 24, reference numeral 32 refers to the threads inside the base of the cap 34 that is twisted within the threads 26 on the outside of the top side 22 (FIG. 1).

The embodiment in FIG. 1 illustrates the present invention 12 in the form of a standard 0.65 FL.OZ/19 mL sized plastic dropper bottle. It is to be understood, however, that the embodiment shown in the drawing is merely an illustration, and that the present invention 12 is applicable to other types and configurations of eye droppers and/or plastic dropper bottles.

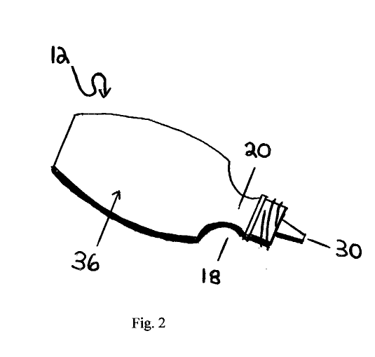

The back 36 of a plastic dropper bottle of the present invention shown in FIG. 2 (shown as a standard sized plastic dropper bottle), is in a application position slightly angled, the process involves a user to tilt the head back and to rest the plastic dropper bottle 12 on the bridge of the nose within either indentation 18, and to position the dropper 30 above the eye without touching the eye, enabling a user to dispense a ophthalmic solution in either eye easily, safely, efficiently and conveniently with a steady hand, independently; a application angle may vary for different users (FIG. 2). It is to be understood that the form of the plastic dropper bottle shown in the drawing is for purposes of illustration only, and that other forms of eye droppers and/or plastic dropper bottles are suitable for use as part of the present invention.

In this embodiment, each indentation preferably has a diameter between approximately 8 mm, and most preferably the distance between the center points of the two indentations is between approximately 16 mm leaving a gap 20 between approximately 8 mm between the indentations (FIG. 2).

The two indentations 18 molded within the plastic dropper bottle of the present invention is sufficient to enable one to rest the plastic dropper bottle 12 on the bridge of the nose within either indentation 18, and to position the dropper 30 above the eye without touching the eye, further enabling one to dispense a ophthalmic solution in either eye with a steady hand, independently (FIG. 2). The plastic dropper bottle of the present invention can be manufactured using conventional processes, with the indentations molded within, using conventional equipment.

In general the plastic dropper bottle 12 of the present invention can be manufactured using conventional methods of aseptic manufacturing, this aseptic manufacturing process refers to manufacturing and packaging of sterile liquids wherein the formation of the container is filling with liquid and formation of the seal to the container is achieved aseptically, this process involves forming the primary container easy drop 12 by heating a thermoplastic polymeric, polyethylene or polypropylene granule to a temperature which is sufficient to mold easy drop 12 which is then filled with the desired ophthalmic solution and then finally sealed, all in a clean and controlled environment. The material can be molded in the configuration shown in FIG. 1 or other types of eye droppers and/or plastic dropper bottle configurations that can be manufactured.

The purpose of the invention is to enable a user to rest the plastic dropper bottle on the bridge of the nose within either indentation 18, and to position the dropper 30 above the eye without touching the eye, further enabling a user to dispense a ophthalmic solution in either eye with a steady hand (as shown in FIG. 2), independently; it is to be understood that a application angle may vary for different users.

Thus, the plastic dropper bottle of the present invention enables its users to rest the plastic dropper bottle 12 on the bridge of the nose within either indentation 18, further enabling a user of the present invention to dispense a ophthalmic solution in either eye with a steady hand, independently (FIG. 2). In recent years concerns about dispensing ophthalmic solutions has been heightened, resulting in numerous new developments; with respect to the prior art, there is not a way for an individual to dispense ophthalmic solutions safely and efficiently without using an extra device or placing a device on the eye or to close to the eye in which such a device could be infectious and/or dangerous to a user's eye. The use of the present invention should provide its users with the convenience of dispensing a ophthalmic solution in either eye with a steady hand, easily, safely and efficiently, without using an extra device or placing a device on the eye or to close to the eye in which such a device could be infectious and/or dangerous to a user's eye.

The present invention offers its users the advantage of dispensing a ophthalmic solution in either eye with a steady hand, without using an extra device or placing a device on the eye or to close to the eye in which such a device could be dangerous and/or infectious to a user's eye.

Therefore, although this invention has been described with a certain degree of particularity, it is to be understood that the present disclosure has been made only by way of illustration and that numerous changes in the details of construction and arrangement of parts may be resorted to without departing from the spirit and scope of the invention.

1. Easy drop comprising:
a body made of a plastic material, the body including a front side and a back side, a bottom side, a top side and two side ends, a dropper that is attached in the top side within the aperture, including a cap that is twisted within the threads on the outside of the top side over the dropper to assure the ophthalmic solution remains in the plastic dropper bottle when not in use; and
two indentations molded within, each indentation is molded within each top portion of each side end and within the top portions of the front side and back side;
Such that easy drop will enable a user to rest the plastic dropper bottle on the bridge of the nose within either indentation, and to position the dropper above the eye without touching the eye, further enabling a user to dispense a ophthalmic solution in either eye with a steady hand, independently.

2. Easy drop as described in claim 1, wherein the two indentations are molded within the top portions of the two side ends.

3. Easy drop as described in claim 2, wherein the two indentations are within the top portions of the front side and back side.

4. Easy drop as described in claim 3, wherein the two indentations have a diameter between approximately 2 and 12 mm.

5. Easy drop as described in claim 4, wherein the two indentations preferably have a diameter between approximately 8 mm.

6. Easy drop as described in claim 5, wherein the two indentations preferably are separated by between approximately 2 and 12 mm.

7. Easy drop as described in claim 6, wherein the two indentations most preferably are separated between approximately 8 mm

8. A device to enable a user to rest the plastic dropper bottle on the bridge of the nose within either indentation, and to position the dropper above the eye without touching the eye, further enabling a user to dispense a ophthalmic solution in either eye with a steady hand, independently, the device comprising:
a dropper bottle formed from a plastic material, the plastic dropper bottle includes a front side and backside, a bottom side, a top side and two side ends, a dropper that is attached in the top side within the aperture, including a cap that is twisted within the threads on the outside of the top side over the dropper to assure the ophthalmic solution remains in the plastic dropper bottle when not in use; and
a means for a user to dispense a ophthalmic solution in either eye with a steady hand, independently, wherein the means for dispensing the ophthalmic solution includes a indentation molded within each top portion of each side end, and within the top portions of the front side and back side;
thereby enabling a user to rest the plastic dropper bottle on the bridge of the nose within either indentation, and to position the dropper above the eye without touching the eye, further enabling a user to dispense a ophthalmic solution in either eye with a steady hand, independently.

9. The device as described in claim 8, wherein the two indentations are molded within the top portions of the two side ends.

10. The device as described in claim 9, wherein the two indentations are within the top portions of the front side and back side.

11. Easy drop comprising:
a body made of a plastic material, the body including a front side and a back side, a bottom side, a top side and two side ends, a dropper that is attached in the top side within the aperture, including a cap that is twisted within the threads on the outside of the top side over the dropper to assure the ophthalmic solution remains in the plastic dropper bottle when not in use; and
two indentations molded within, each indentation is molded within each top portion of each side end and within the top portions of the front side and back side, the two indentations preferably have a diameter between approximately 8 mm, and the two indentations preferably are separated between approximately 2 and 12 mm;
such that easy drop will enable a user to rest the plastic dropper bottle on the bridge of the nose within either indentation, and to position the dropper above the eye without touching the eye, further enabling a user to dispense a ophthalmic solution in either eye with a steady hand, independently.

12. Easy drop comprising:
a body made of a plastic material, the body including a front side and a back side, a bottom side, a top side and two side ends, a dropper that is attached in the top side within the aperture, including a cap that is twisted within the threads on the outside of the top side over the dropper to assure the ophthalmic solution remains in the plastic dropper bottle when not in use; and
two indentations molded within, each indentation is molded within each top portion of each side end and within the top portions of the front side and back side, the two indentations preferably have a diameter between approximately 8 mm, and the two indentations preferably are separated between approximately 2 and 12 mm;
such that easy drop will enable a user to rest the plastic dropper bottle on the bridge of the nose within either indentation, and to position the dropper above the eye without touching the eye, further enabling a user to dispense a ophthalmic solution in either eye with a steady hand, independently.
